(19) United States
(12) Patent Application Publication
(10) Pub. No.: US 2026/0365981 A1
(43) Pub. Date: Nov. 19, 2026

(54) METHODS FOR HEMATOPOIETIC STEM AND PROGENITOR CELL EXPANSION AND DIFFERENTIATION IN THREE-DIMENSIONAL SILK SCAFFOLDS AND USES THEREOF

(71) Applicant: UNIVERSITÀ DEGLI STUDI DI PAVIA, Pavia (IT)

(72) Inventors: Alessandra BALDUINI, Pavia (IT); Christian Andrea DI BUDUO, Chignolo Po (PV) (IT)

(21) Appl. No.: 19/102,145

(22) PCT Filed: Aug. 8, 2023
(86) PCT No.: PCT/EP2023/071983

§ 371 Date: Feb. 7, 2025

(30) Foreign Application Priority Data
Aug. 10, 2022 (IT) 102022000017079

(51) Int. Cl. C12N 5/078

(52) U.S. Cl. CPC C12N 5/0641; C12N 5/0644; C12N 2501/165; C12N 2501/21; C12N 2502/1311; C12N 2502/1352; C12N 2506/11; C12N 2513/00; C12N 2533/50; C12N 2533/52; C12N 2537/00; C12N 2539/00

(57) ABSTRACT

The present invention relates to silk fibroin-based scaffolds suitable for supporting the 3D culture of human hematopoietic stem and progenitor cells and/or the production of blood cells ex-vivo, and methods for producing the same.

### ABSTRACT

The present invention relates to silk fibroin-based scaffolds suitable for supporting the 3D culture of human hematopoietic stem and progenitor cells and/or the production of blood cells ex-vivo, and methods for producing the same.

### BACKGROUND

Hematopoiesis occurs in the bone marrow. Hematopoietic stem and progenitor cell (HSPC) fate decisions are dependent on signals from specialized microenvironments in the bone marrow, termed niches, three-dimensional (3D) environments that comprise cellular, chemical, and physical elements.

Most experimental approaches to study human hematopoiesis use bidimensional culture techniques with poor control over cell stemness and differentiation capability. Tissue-engineering approaches have been developed to create 3D-functional mimics of the native organs (Di Buduo et al., 2021 [1]). The bone marrow represents a challenging tissue to reproduce because its structure and composition confer unique biochemical and mechanical features to control HSPC quiescence, expansion, and differentiation.

Reproducing the human bone marrow niche is instrumental to answer the growing demand for human blood cells production ex-vivo either for fundamental studies or for clinical applications in the fields of transfusion and regenerative medicine, and bone marrow transplantation:
models that recapitulate the complexity of human bone marrow can foster mechanistic studies of normal and malignant hematopoiesis and the validation of novel pharmacological therapies.
HSPCs transplantation is the leading clinical application of cell-based therapies. Successful transplantations require a considerable amount of high-quality HSPCs to reconstitute long-term hematopoiesis. However, present-day procedures for harvesting and manipulating these cells cannot guarantee optimal therapeutic yield nor the quality of transplantable HSPCs. HSPC processing in conditions that reproduce their native bone marrow environment may lead to improvements in HSPC function and engraftment outcomes.
more than 100 million units of blood are reported to be collected worldwide every year. Nevertheless, in no country does the contribution of volunteers succeed in coping with the growing demand, making it necessary to create alternative methods to produce blood cells. Ex-vivo manufacturing of mature blood cell products (i.e., erythrocytes and platelets) is becoming an increasingly attractive approach for both basic research and clinical applications (e.g., transfusion medicine).

In vitro, culture conditions miss the bone marrow's physical environment, which drives either HSPC self-renewal or differentiation into mature blood cells in vivo. In most of the current in vitro culture systems, HSPCs lose stemness a few hours after starting the culture, while differentiated blood cells look immature. For example, erythrocytes appear macrocytic, and platelets appear larger with immature granules. Also, it is known that cultured erythrocyte progenitors fail the enucleation process, while megakaryocytes of any origin produce fewer platelets per single cell than they do in vivo [1].

3D bone marrow organoids have been developed, though these systems allow the development of a self-made bone marrow microenvironment useful for mechanistic studies with limited possibility of experimental control and hampered cell harvesting and usage for clinical application (Khan et al., 2022 [2]).

3D scaffolds made of different types of organic (e.g., PEG, PCL, PU) and inorganic (e.g., HAp) biomaterials have been proposed to reproduce the 3D architecture as well as mechanical properties, nanopatterning, and topography of the native bone marrow. Also, their biofunctionalization through surface coatings, absorption, and/or covalent conjugation of ECM-derived molecules or cell-interacting motifs (e.g., collagen, fibronectin, laminin) has been proposed. Though, they could only support targeted HSPCs functions and concise experiments that hindered the possibility of transferring a single technique/approach into wide applications [1].

Shear forces occurring by fluid flow and gradients of soluble factors, such as growth factors or cytokines, play a role in controlling HSPC behavior. The emergence of bone marrow-on-chip technologies has empowered the development of more complex models of the niche. These systems were able to provide a hydrodynamic shear supporting dynamic cell culture, but with low efficiency in terms of cell numbers because of their nano/micro-scale nature which cannot support large-scale approaches (e.g., industrial application, implementation into clinical processes).

Overall, none of the available systems have been validated for supporting at the same time long-term HSPC culture and/or their programmable differentiation into different blood lineages.

For applications aiming to mimic different natural processes of the niche, the complexity of the system must be reduced to the essentials to be easily modeled according to the scientific question to be addressed.

In the past 10 years, the present inventors' expertise in modeling silk fibroin biomaterial has led to the development of early models of silk-bone marrow able to produce human platelets by in vitro differentiated megakaryocytes from cord blood-derived HSPCs to provide proof-of-concept evidence of their applicability for transfusion medicine.

The first generation of silk-based bone marrow models developed by the inventors (Pallotta et al., 2011 [3]; Di Buduo et al., 2015 [4], international patent application WO2016/022834 A1 [5]) were composed of porous silk tubes, mimicking vessels, surrounded by a 3D matrix to allow the recording of megakaryocyte functions (i.e., migration, proplatelet formation) in response to variations in extracellular matrix components, surface topography and stiffness, and co-culture with endothelial cells. This method could produce human platelets, but not other blood cells, and cannot support long-term cultures. This system demonstrated the fundamental qualities of silk fibroin for studying thrombopoiesis, such as non-thrombogenicity and the possibility to entrap different molecules while retaining their bioactivity. A limitation of this model was the use of custom-made chambers and silk tubes whose production could not be standardized or scaled up easily to guarantee clinical applications.

The second generation of silk-bone marrow models developed by the inventors (Di Buduo et al., 2017 [6], Tozzi et al., 2018 [7]) consisted of a scaled-up version intended to house a larger number of in vitro-derived megakaryocytes producing platelets for functional studies. The flow chambers were made of research-grade silicon or biomaterial, holding a silk sponge, prepared with salt leaching methods [6] or by lyophilization [7], and functionalized with extracellular matrix components (fibronectin [6], collagen IV [7]). Perfusion of the chambers allowed the recovery of platelets when the silk sponges were cultured with cord blood-derived megakaryocytes.

A miniaturized system (Di Buduo et al., 2021 [8]; international patent application WO2021/113830A1 [9]) could produce measurable numbers of platelets by megakaryocytes differentiated in vitro by HSPC derived from adult peripheral blood.

None of these systems was designed to produce blood cells other than platelets and cannot support long-term cultures. Indeed, all these systems have been validated by culturing megakaryocytes for a few hours ([3], [4], [6], [7]) or a few days [8]. HSPC differentiation into megakaryocytes was carried out in classic Petri dishes.

As a result, the efficiency of the systems was limited because:
prior art methods could not support long-term HSCPs expansion and retrieval or differentiation into different blood cell lineages.
prior art methods could not support simultaneous long-term co-culture of HSPCs with other non-hematopoietic cell types (e.g., mesenchymal stem cells, osteoblasts, osteoclasts, osteocytes, fibroblasts, endothelial cells, perivascular cells, adipocytes, neurons).
prior art methods could not support simultaneous long-term co-culture of HSPCs with mature blood cells (e.g., leukocytes, granulocytes, monocytes, lymphocytes, neutrophils, eosinophils, basophils, macrophages, erythrocytes, megakaryocytes, and platelets).
prior art methods could not allow control over oxygen distribution within the culture.

In view of the above drawbacks, the present invention provides 3D silk scaffolds and methods for:
human HSPC culture and expansion while keeping stemness in 3D silk scaffold. The culture method allows cell recovery for performing functional studies (e.g., colony formation assays, in situ differentiation).
human HSPC differentiation into red blood cells. The culture methods allow the spontaneous formation of erythroblastic islands that support final erythrocyte maturation and enucleation.
human HSPC differentiation into platelet-forming megakaryocytes. The improved culture method allows the production of an increased number of platelets with improved functionality with respect to previous methods and 2D liquid culture.
production of multi-layer scaffolds for the co-culture of HSPCs with mesenchymal stem cells, osteoblasts, osteoclasts, osteocytes, fibroblasts, endothelial cells, perivascular cells, adipocytes, neurons, leukocytes, granulocytes, monocytes, lymphocytes, neutrophils, eosinophils, basophils, macrophages, erythrocytes, platelets, megakaryocytes alone or in combination, to support improved HSPC differentiation. The stiffness of the silk scaffold to guide cell function may be regulated.
silk scaffold perfusion into programmable flow chambers with a culture medium at different flow rates and timing (e.g.; continuous, intermittent). The composition of the perfused medium can be varied to guide HSPC quiescence, expansion or differentiation.

### SUMMARY OF THE INVENTION

The present invention relates to a 3D silk fibroin scaffold for HSPC culture and differentiation into mature blood cells. A multi-layer scaffold can be assembled comprising at least two interconnected silk scaffolds:
i) an inner core made of a porous solid silk fibroin scaffold or of a silk fibroin hydrogel comprising stem cells of the hematopoietic lineage;
ii) at least one silk fibroin scaffold surrounding the inner scaffold i) made of silk fibroin hydrogel or porous solid silk fibroin scaffold comprising differentiated or undifferentiated cells selected between hematopoietic stem cells, or hematopoietic progenitor cells, mature blood cells and/or other non-hematopoietic cell types (e.g., mesenchymal stem cells, osteoblasts, osteoclasts, osteocytes, fibroblasts, endothelial cells, perivascular cells, adipocytes, neurons).

Solid and hydrogel silk scaffolds could be assembled into 3D multi-layer tissues that mimic the structure and composition of the different bone marrow microenvironments. The minimum number of layers is two. As many layers as needed can be added based on the experimental needs.

Silk fibroin can be of a natural origin, such as, for example, fibroin produced by arthropods such as silkworms (e.g., Bombyx mori, Anthereae pernyi) or spiders (e.g., Nephila clavipes, Araneus diadematus). Alternatively, the silk fibroin can be of a recombinant origin, such as for example the fibroin produced by engineered systems (e.g.; bacteria, yeast). Alternatively, fibroin can be reconstituted from a lyophilized formulation. Silk fibroin can be chemically modified. According to a preferred embodiment, silk fibroin is extracted from Bombyx mori silkworm cocoons.

In some embodiments, resulting silk-based scaffolds can be arranged into pre-determined patterns (e.g., cylindrical, rectangular, spherical, hexagonal) for controlled localization of at least two different cell types within the multi-layer scaffold.

In a preferred embodiment of the 3D silk fibroin multi-layer scaffold of the invention, the inner scaffold comprises stem cells of the hematopoietic lineage. According to another embodiment of the 3D silk fibroin multi-layer scaffold according to the invention, the undifferentiated cells of the silk fibroin scaffold ii) are stem and progenitor cells of the hematopoietic lineage, mesenchymal stem cells, endothelial progenitor cells, neural stem cells, induced pluripotent stem cells or embryonic stem cells, when the 3D silk fibroin multi-layer scaffold further comprises one or more additional silk layer.

In a preferred embodiment, said stem cells of the hematopoietic lineage are HSPCs. In another preferred embodiment, said stem cells of hematopoietic lineage are derived from induced pluripotent stem cells. In a further preferred embodiment said stem cells of hematopoietic lineage are derived from embryonic-derived cells.

In a preferred embodiment, the solid silk scaffold, the hydrogel scaffold, or at least one of the two layers of the multi-layers scaffold comprises a cell component selected in the range of 0.01-100×10³ cells/mm³.

In a preferred embodiment, the silk scaffold volume is a minimum of 3 mm³. In another preferred embodiment, the other layer (or layers) ii) comprises differentiated cells belonging to the category selected among mammalian osteoblasts, osteoclasts, osteocytes, fibroblasts, endothelial cells, perivascular cells, adipocytes, neurons, leukocytes, granulocytes, monocytes, lymphocytes, neutrophils, eosinophils, basophils, macrophages, erythrocytes, platelets, megakaryocytes and/or undifferentiated cells belonging to the category selected among stem and progenitor cells of the hematopoietic lineage, mesenchymal stem cells, endothelial progenitor cells, neural stem cells, induced pluripotent stem cells or embryonic stem cells, in the range of 0.1-100×10³ cells/mm³.

In another preferred embodiment, at least two different cell types can be co-cultured in the same or different layer.

According to a preferred embodiment of the 3D silk fibroin multi-layer scaffold of the invention, the porous solid silk fibroin scaffold comprises interconnected pores having a diameter>5 μm. The porosity ensures the exchange of soluble factors and the possibility of cell migration among different compartments.

The scaffolds according to the present invention were tested by culturing different cell types, such as human blood cancer cell lines, human HSPCs, murine HSPCs, human induced pluripotent stem cells, human and murine bone marrow cells, human and mouse mature blood cells, thus showing a wide versatility of use. Before seeding HSPCs, the silk fibroin scaffolds and hydrogels, alone or in combination, can be enriched with a different combination of differentiated cells belonging to the category selected among mammalian osteoblasts, osteoclasts, osteocytes, fibroblasts, endothelial cells, perivascular cells, adipocytes, neurons, leukocytes, granulocytes, monocytes, lymphocytes, neutrophils, eosinophils, basophils, macrophages, erythrocytes, platelets, megakaryocytes and/or undifferentiated cells belonging to the category selected among stem cells of the hematopoietic lineage, mesenchymal stem cells, endothelial progenitor cells, neural stem cells, induced pluripotent stem cells or embryonic stem cells, (alone or in different combinations).

The elasticity and viscosity of silk fibroin can be adjusted depending on the methods used for the fibroin extraction.

Oxygen distribution inside the scaffold can be controlled by varying silk fibroin composition and assembly at the surface of the scaffolds and/or hydrogels.

To obtain the solid silk scaffold NaCl₂ particles (approximately >300 μm in diameter) were sifted into a silk solution (2-20% w/v) in a ratio of 1 mL silk/2 g salt, within a molding system (e.g., petri dish, silicon chamber). The scaffolds were then placed at room temperature for 48 hours and then soaked in distilled water for 48 hours to leach out the salt particles. The scaffolds were sterilized in 70% ethanol and finally rinsed five times in phosphate-buffered saline (PBS) over 24 hours.

Alternatively, the silk solution (1-20% w/v) was cast within the molding system and incubated at −80 °C for one day. The frozen solution was then lyophilized for 48-72 hours at −56/−80 °C. The lyophilized scaffolds were subsequently autoclaved for 20 minutes to induce the β-sheet formation and stabilize the silk matrix.

Both types of silk scaffolds were characterized by confocal microscopy and scanning electron microscopy. Depending on silk fibroin degumming time (10, 20, 30, 40, 50, or 60 minutes) and final concentration (1-20%), and on β-sheet inducing parameters (salt leaching or lyophilization), the bulk region of these solid scaffolds can be prepared with control over a variety of factors, including, but not limited to, pore sizes and morphologies, mechanical properties, degradation rates, and any combinations thereof.

The hydrogel silk scaffold is composed of:

| | | |
|---|---|---|
| silk fibroin | 0.5%-35% | weight/volume (w/v) |
| gelatin or derivatives thereof | 5-20% | w/v |
| alginic acid or derivatives thereof | 0.5-2% | w/v |
| glucose or analogues thereof | 3-4% | w/v |
| controlled density solution | 1-3% | w/v |
| buffer | 4-6% | w/v |
| albumin | 0.01-1% | w/v |

| | |
|---|---|
| saline buffer solution | up to 100% |

wherein the gelatin and alginic acid or their derivatives are present in a reciprocal ratio ranging from about 10:1 to about 20:1; wherein the silk fibroin and the gelatin are present in a reciprocal ratio ranging from 15 about 1:5 to about 1:0.5; suitable for mixing with hematopoietic cells. The cell component can be part of the initial formulation. The temperature of the silk hydrogel formulation is 37 °C when the cells are to be added.

Silk hydrogels were produced at 37 °C. After deposition, the silk hydrogel could be crosslinked with a CaCl₂) solution for a minimum of 10 minutes. The crosslinking solution preferably contains 0.05-0.1 M CaCl₂) dissolved in a buffer solution containing NaCl, KCl, MgCl₂, glucose, and HEPES.

In a preferred embodiment, the saline buffer solution contains: 150 mM NaCl, 6 mM KCl, 1 mM MgCl₂, 10 mM HEPES.

In a preferred embodiment of the silk fibroin hydrogel formulation according to the invention, the gelatin and alginic acid or their derivatives are present in a reciprocal ratio of about 15:1.

Again, in accordance with a preferred embodiment of the silk fibroin hydrogel formulation according to the invention, the fibroin and gelatin or its derivatives are present in a reciprocal ratio equal to about 1:5 or about 1:2 or 1:0.5.

Optionally, the silk fibroin is dissolved in a solution comprising one or more ion sources selected from the group consisting of alkaline or alkaline earth metal 10 chlorides, such as for example MgCl₂, CaCl₂), NaCl, KCl.

The gelatin used in the silk fibroin hydrogel formulation according to the invention can be type A (acid hydrolysis) or type B (alkaline hydrolysis). Preferably, gelatin is of type A.

According to a preferred embodiment of the formulation according to the invention, the gelatin derivatives are selected from the group consisting of gelatin conjugated with chitosan, gelatin-poly(DL-lactide), gelatin modified with PEG, gelatin thiolates, DNA-gelatin nanospheres, gelatin nanoparticles, gelatin-coated fluorescent maghemite nanoparticles, gelatin-coated fluorescent polymethacrylic acid nanoparticles (FPMAAG), supramolecular gelatin nanoparticles coated with quantum dots, gelatin nanoparticles coated with iron oxide, gelatin methacrylate, xanthan gum human gelatin, recombinant gelatin, gelatin marked with fluorescent molecules.

According to a preferred embodiment of the formulation, the alginic acid derivatives are salts with alkaline and alkaline earth metals selected from the group consisting of sodium alginate, calcium alginate, magnesium alginate, and potassium alginate. Alternatively, methacrylate alginate can be used to produce photopolymerizable hydrogels in the presence of soluble photoinitiators. According to preferred embodiments of the invention, the glucose analogues are selected from the group consisting of disaccharides, stereoisomers, isomers, epimers, alditols or acids of glucose, precursors or products deriving from the glucose metabolism. Glucose analogues within the context of the present invention refer to metabolites that can be transported within the cell, become part of the glycolysis cycle, and metabolized.

Other sugars, such as xylose, do not form part of the present invention as they are not capable of entering the cell via glucose transporters and cannot enter the glycolysis cycle. The cells are therefore not capable of metabolizing them.

By way of example, the glucose analogues can be selected from the group consisting of: D-glucose, L-glucose, lactose, sucrose, trehalose, cellobiose, melibiose, maltose (disaccharides), mannitol, galactose, mannose, fructose (epimers), allose, altrose (stereoisomers), gluconic acid, glucuronic acid (acids), arabinose, glycerol, pyruvate, glucose 1-phosphate, glucose 6-phosphate, lactate, malate, phosphoglycerate, succinate (precursors or derivatives of the glucose metabolism), sorbitol (alditol). The glucose can also be replaced by fluorescent analogues that allow the glucose transportation to be monitored (e.g. 2-(N-(7-nitrobenz-2-oxa-1,3-diazol-4-yl)amino)-2-deoxyglucose) or analogues that inhibit the glycolytic metabolism (e.g. 2-deoxy-d-glucose).

In further preferred embodiments of the invention, the controlled density solution is a controlled density solution selected from the group consisting of Percoll® (colloidal silica particles of 15-30 nm in diameter (23% w/w in water) coated with polyvinylpyrrolidone), Ficoll® (sucrose and epichlorohydrin copolymer), colloidal silica, copolymers (e.g. sucrose and epichlorohydrin), hydrophilic polysaccharides (e.g. sucrose), high-molecular-weight synthetic polymers (e.g. Polysucrose®). The controlled density solution is preferably Percoll®.

The choice of the controlled density solution depends on the degree of density to be attributed to the formulation for optimizing the distribution of cells in the suspension and eventually preserving their vitality during the printing process. Further, preferred embodiments of the hydrogel formulation according to the invention provide that the buffer be an organic or mineral buffer selected from the group consisting of: MES, ADA, ACES, PIPES, MOPSO, Bis-6Tris Propane, BES, MOPS, TES, HEPES, DIPSO, MOBS, TAPSO, HEPPSO, POPSO, EPPS, Tricine, Gly-Gly, Bicine, HEPBS, TAPS, AMPD, TABS, AMPSO, CHES, CAPSO, AMP, CAPS and CABS. The buffer is preferably HEPES (4-2-hydroxyethyl-1-piperazinyl-ethanesulfonic acid) which has strong buffering power.

Based on the culture conditions required for ensuring vitality and functionality of the cell type being mixed with the hydrogel, the ideal buffer system is selected that allows the desired pH, the subsequent culture, and in any type of manipulation and/or analysis of the scaffolds in an uncontrolled CO₂ atmosphere.

The presence of the buffer, for example, is an advantage during the deposition process. In this way, it is possible to keep the cells viable as they are maintained at a physiological pH, even outside of the incubator.

The silk hydrogel formulation according to the invention always maintains a physiological pH. Without a buffer, the formulation can lose its physiological pH and this results in a reduction in cell vitality/functionality.

In a preferred embodiment, the formulation according to the invention has the following composition:

| silk fibroin | 8% | w/v |
|---|---|---|
| gelatin | 15% | w/v |
| alginic acid | 1% | w/v |
| glucose | 3.5% | w/v |
| Percoll ® | 2.5% | w/v |
| HEPES | 5% | w/v |
| saline buffer solution | up to 100% | |
| albumin | 0.1% | w/v |

Solid and hydrogel silk scaffolds could be assembled into 3D multi-layertissues that mimic the structure and composition of the different bone marrow microenvironments. The minimum number of layers is two. As many layers as needed can be added based on the experimental needs.

According to a preferred embodiment, the central core is a porous solid silk scaffold obtained by salt leaching processing of 50/60-minute degummed silk (6-8%) or by lyophilizing 30/60-minute degummed silk (2-6%). The second layer of the tissue model is fabricated with salt leaching methods using 10/30-minute degummed silk fibroin. Alternatively, the first silk scaffold is covered by a silk hydrogel according to the composition mentioned above. The silk hydrogel solution is dispensed at 37 °C around the first solid silk scaffold, and then crosslinked at room temperature. According to a preferred embodiment, silk fibroin hydrogel is dispensed by manual pipetting. Alternatively, the hydrogel can be bio-printed around the solid silk fibroin scaffold through an extrusion-based approach with a nozzle having a caliber ranging from 18G to 27G, extrusion pressure within the range of 5-200 kPa, and printing rate within the range of 5-1,000 mm/min.

According to a preferred embodiment the multi-layer model, comprise at least two interconnected silk scaffolds, wherein at least one scaffold partially surrounds the other, and at least one of the scaffolds comprises interconnected pores, wherein pores have a diameter>5 μm.

The present invention further relates to the use of the scaffolds described above as an ex-vivo model for keeping stemness, supporting hematopoiesis or for the production of mature blood cells (including erythrocytes, macrophages, megakaryocytes, leukocytes, granulocytes, lymphocytes, and platelets) through the use of growth factors or mediums that maintain the stemness or promote cell differentiation. The cells retrieved from the scaffolds may be used for morphological, functional characterization (e.g., expression of lineage-differentiation markers; differentiation assays), and clinical applications (e.g., transfusion, transplant).

Further, the invention relates to the use of the scaffold described above as a surgical implant for keeping stemness, supporting hematopoiesis or for the production of erythrocytes, macrophages, megakaryocytes, leukocytes, granulocytes, lymphocytes, platelets, and other blood cells in vivo.

In a preferred embodiment of the invention, said implant further comprises one or more drugs, hormones, growth, or differentiation factors that can be released in situ.

In some embodiments, the silk fibroin scaffolds, and hydrogels of the invention can be functionalized with bioactive molecules (i.e., enzymes, substrates and intermediates of enzymatic reactions, extracellular matrices, drugs, cytokines, chemokines, growth factors, hormones, proteins, and glycoproteins, cell-derived interacting motifs, biologic fluids).

In particular, this bioactive molecule may be selected among the group consisting of:
components of the extracellular matrix selected from the group consisting of proteoglycans, hyaluronic acid, collagens, elastin, fibronectin, fibrin, fibrinogen, laminins, thrombospondin;
growth factors, cytokines, and chemokines, interleukins, CSF-1, G-CSF, M-CSF, GM-CSF, SCF, FLT3-L, TPO, EPO, SDF-1α; TGF-β1, TNFα, VEGF, FGF, Notch ligands, WNT, angiopoietin-1, BMP, IGF-2 and fragments or variants thereof;
cellular secretome and cell-derived growth factors (e.g., platelet secretome, leukocyte secretome, platelet-derived growth factors);
polyol compounds such as glycerol;
plasma proteins or glycoproteins, selected among albumin, globulins, transferrin, immunoglobulins;
lipids, cholesterol and lipoproteins;
biologic fluids such as whole blood, bone marrow aspirate, blood serum or plasma, bone marrow serum or plasma, or cell-culture supernatants;
cell-derived interacting motifs, antigens, conjugated or unconjugated antibodies, or their fragments such as CDRs or epitopes;
hormones, such as insulin, glucagon, triiodothyronine, thyroxine, steroid hormones or their antagonists;
drugs or prodrugs, such as TPO mimetics, TPO receptor agonists, tyrosine kinase receptor agonists, tyrosine kinase receptor inhibitors, Rho kinase inhibitors, kinase inhibitors, receptor antagonists of aryl hydrocarbons (e.g., stemregenin 1), pyrimidoindole derivatives (e.g., UM729, UM171), chemotherapeutic agents, monoclonal antibodies, polyclonal antibodies.
nucleic acids, such as DNA, RNA, siRNA, RNAi and microRNA, plasmids, lentiviruses, CRISPRs;
enzymes, such as horseradish peroxidase, and/or their substrates or intermediates of enzymatic reactions.
toxins.

According to a further embodiment of the present invention, solid or hydrogel silk scaffolds and their combination thereof can further comprise a fluorescent marker, a contrast agent, an enzyme and/or enzymatic reaction intermediates, a luminescent substance, a chemiluminescent substance, a radio-opaque agent, a radioactive element or a conjugated or unconjugated antibody.

According to an alternative embodiment, the 3D silk fibroin multi-layer scaffold according to the invention further comprises a thin coating layer of silk fibroin surrounding the outer scaffold to control oxygen distribution.

The invention further relates to the use of the 3D silk fibroin monolayer or multilayer scaffold according to the invention as ex-vivo model for supporting hematopoiesis or for the production of mature blood cells (e.g.; erythrocytes, macrophages, megakaryocytes, leukocytes, granulocytes, lymphocytes, platelets, etc.) by expansion and differentiation of HSPC or induced pluripotent stem cells or embryonic stem cells.

According to another aspect the present invention relates to the use of the 3D silk fibroin monolayer or multilayer scaffold of the invention as a surgical implant for supporting hematopoiesis or the production of mature blood cells in vivo. Said mature blood cells are preferably erythrocytes or platelets.

A further object of the invention is a method for expansion and/or differentiation of HSPCs or induced pluripotent stem cells wherein the 3D silk fibroin monolayer or multiplayer scaffold of the invention is cultured in plate in static conditions or perfused into dynamic flow chambers with a cell culture medium comprising at least one nutrient (e.g., glucose), cytokine, growth factor, hormone, drug or prodrug, biologic fluid, plasma protein, plasma glycoprotein or a combination thereof. In some embodiments, HSPCs can be retrieved from the silk scaffold.

The methods to expand and differentiate the HSPCs of the invention may be carried out in static and dynamic culture conditions.

In some embodiments, the 3D silk scaffolds can be cultured into a perfusion chamber. In some embodiments, the flow-through contains cytokines, growth factors, cell-derived secretome, hormones, drugs or prodrugs, biologic fluids, plasma proteins, plasma glycoproteins or a combination thereof to support differently HSPC expansion and/or differentiation. In some embodiments, the flow rate can be modified to control the distribution of oxygen and nutrients, and shear stress inside the chamber.

In some embodiments, perfusion is performed at shear stresses of 0.05-50 dyn/cm².

In some embodiments, the perfusion can be continuous, or intermittent (run and stop). In some embodiments, the dynamic culture can be live-imaged during the perfusion.

### HSPC Expansion

CD34⁺ HSPCs were obtained from human cord blood or human peripheral blood (purity>90%), and seeded at the concentration of 0.5-5×10³ cells/mm³ in serum-free medium supplemented with 1% penicillin/streptomycin (P/S), 1% L-glutamine, 1 μM SR-1, SCF 100 ng/ml, Flt3-L 100 ng/ml, TPO 10 ng/ml. HSPCs were cultured in a 5% CO₂ for at least 14 days in a humidified atmosphere at 37 °C.

### Megakaryocyte Differentiation and Platelet Production

CD34⁺ HSPCs were obtained from adult peripheral blood or cord blood. 2-5×10³ cells/mm³ were seeded into the silk scaffolds. Samples were differentiated for 14 days in serum-free medium supplemented with 1% P/S, 1% L-glutamine, 10 ng/mL TPO, 10 ng/mL IL-11, at 37 °C in a 5% CO₂ fully humidified atmosphere.

### Erythrocyte Differentiation and Red Blood Cell Production

CD34⁺ HSPCs were obtained from adult peripheral blood. 2-5×10³ cells/mm³ were seeded into the silk scaffolds. Samples were differentiated for at least 14-21 days in serum-free medium supplemented with 1% P/S, 1% L-glutamine, 20 ng/mL EPO, 10 ng/mL IL-3, 800 ng-800 μg/mL holo-transferrin, at 37 °C in a 5% CO₂ fully humidified atmosphere.

### Mature Blood Cell Culture

Mature megakaryocytes, erythrocyte, macrophages or leukocytes were seeded into the silk scaffolds and cultured, at 37 °C in a 5% CO₂ fully humidified atmosphere.

### Bone Marrow Cell Culture

Human bone marrow mesenchymal stem cells (MSCs) were cultured in high glucose medium supplemented with sodium pyruvate, 10% MesenCult™ MSC Stimulatory Supplement (Human), 1% L-glutamine, 1% P/S, and 1% non-essential amino acids. MSCs were seeded at a density of 1-2×10³ cells/mm³. MSCs were cultured for at least 7 days, before starting the co-cultures.

Osteoblasts were differentiated from human bone marrow MSCs in high glucose medium supplemented with sodium pyruvate, 10% fetal bovine serum (FBS), 1% L-glutamine, 1% P/S, 1% non-essential amino acids, 100 nM dexamethasone, 10 mM sodium β-glycerol phosphate and 50 μM ascorbic acid. The MSCs were left to differentiate into osteoblasts for at least 3 weeks, before starting the co-cultures.

Adipocytes were differentiated from human bone marrow MSCs in high glucose medium supplemented with sodium pyruvate, 1% L-glutamine, 1% P/S, 10% FBS, 0.5 mM isobutyl-methylxanthine, 1 μM dexamethasone, 10 μM insulin, 100 μM indomethacin for three days and maintained in medium with 10% FBS and 10 μM insulin for one day. The treatment was repeated three times, after which the cells were maintained in high glucose medium supplemented with 10% FBS and 10 μM insulin until day 21.

Human umbilical vein endothelial cells (HUVEC), were cultured in medium supplemented with 5% fetal bovine serum, 1% P/S, hEGF, hydrocortisone, GA-1000, BBE, ascorbic acid. HUVEC were seeded at a density of 1-2×10³/mm³ and cultured for at least 7 days, before starting the co-cultures.

In some embodiments, the present invention provides methods for highly customizable models of the human bone marrow characterized by their ability to produce differentiated blood cells ex-vivo from HSPCs of healthy subjects or donors with hematological diseases (e.g., genetic diseases, hematologic cancers, etc.). HSPCs can be from cord blood or peripheral blood or bone marrow and can be genetically modified either before or during the 3D culture.

It has been observed that, i.e. the production of platelets by using the 3D silk fibroin scaffolds of the invention in the monolayer or multiplayer format (hydrogel silk scaffold alone or solid-solid silk scaffold in the outer and inner layer of a multilayer silk scaffold of the invention) is at numbers at least an order of magnitude above that achieved by known methods/scaffolds.

The methods to expand and differentiate HSPCs by the silk scaffold of the invention may foresee an additional step of retrieval of the cells from the scaffolds, keeping them vital and functional for clinical applications.

In some embodiments, cells that differentiate within the silk scaffold (e.g., mature platelets, mature enucleated erythrocytes) can be retrieved from the system by perfusion into flow chambers at programmable shear stress.

In a preferred embodiment, the formulation of the solution for the retrieval of the whole cell population from the scaffold of the invention consists of sodium citrate, collagenase, proteinase K, liberase and/or alginate lyase, with a source of glucose, comprises:

| sodium citrate | 15 | mM |
|---|---|---|
| collagenase | 10 | U/mL |
| alginate lyase | 5 U/mL (valid for silk hydrogel only) | |
| glucose | 5 | mM |

| | |
|---|---|
| saline buffer solution | up to 100% |

The treatment is performed preferably at 37 °C for 10-20 minutes.

The present invention further relates to the use of cells released from the scaffold for carrying out morphological, functional, biochemical, and/or molecular analyses which can comprise, by way of example, one or more of the following methods:
collection of proteins for proteomics, immunoassays, electrophoresis and/or Western blotting assays;
collection of nucleic acids for genomic characterization, evaluation of the cell karyotype, gene expression analysis via polymerase chain reaction, transcriptomic analyses and/or gene sequencing in all of their variants;
immune characterization of the released cells;
imaging;
enzymatic or colorimetric assays;
flow cytometric analysis and/or selection of specific cell populations;
use of cells for functional assays.

It is another object of the present invention a method for the production of a 3D silk fibroin multilayer scaffold comprising the following steps:
i) obtaining the porous solid silk scaffold by salt leaching processing or by lyophilizing degummed silk;
ii) dispersion of the stem cells of the hematopoietic lineage or progenitor cells into the solid silk scaffold, or into the different layers of solid silk scaffolds, by manual pipetting;
iii) mixing the cells within the silk-based hydrogel before dispensing the solution around, or inside, the solid silk scaffold.

According to a preferred embodiment, the inner core is a porous solid silk scaffold obtained by salt leaching processing of 50/60-minute degummed silk (6-8%) or by lyophilizing 30/60-minute degummed silk (2-6%).

A second layer made of a solid silk scaffold ii) is fabricated with salt leaching methods using 10/30-minute degummed silk fibroin.

Alternatively, the first silk scaffold is covered by a silk hydrogel according to the composition mentioned above. The silk hydrogel solution is dispensed at 37 °C around the first solid silk scaffold, and then crosslinked at room temperature. According to a preferred embodiment, silk fibroin hydrogel is dispensed by manual pipetting. Alternatively, the hydrogel can be bio-printed around the solid silk fibroin scaffold through an extrusion-based approach with a nozzle having a caliber ranging from 18G to 27G, extrusion pressure within the range of 5-200 kPa, and printing rate within the range of 5-1,000 mm/min. Alternatively, the inner core can be the silk hydrogel according to the composition mentioned above with a solid silk scaffold as the second layer.

The method can further comprise a step iv) of forming a coating of silk around the scaffolds, after cell seeding. The coating layer can form a continuous thin silk structure enclosing the scaffold to control oxygen distribution.

The coating layer around the scaffolds can be formed using 0.5-30% w/v silk solution by any methods known to a skilled artisan, for example, without limitation, dipping, spraying, electrospinning, gel-spinning, or any combinations thereof.

In a preferred embodiment, the coating is formed by dipping the scaffolds into a 1-10% w/v silk solution, followed by rapid soaking into dehydrating alcohol (e.g., methanol) and/or water-annealing.

According to a preferred embodiment of the invention, the silk scaffold is sterile. The sterility can be advantageously achieved by means of UV-ray sterilization.

The above stem cells of the hematopoietic lineage are preferably mammalian cells, preferably of human origin. HSPCs or differentiated cells of the hematopoietic lineage can derive directly from human blood, preferably HSPCs are multipotent or pluripotent stem cells. More specifically, they can be hematopoietic stem cells (totipotent or pluripotent, including induced pluripotent stem cells and embryonic stem cells), progenitor cells (such as, for example, progenitors of the myeloid or lymphoid lineage, erythrocytes, megakaryocytes), or differentiated blood cells. The differentiated cells of the myeloid lineage derive from a myeloid progenitor and are selected from the group that comprises monocytes, macrophages, granulocytes, neutrophils, basophils, eosinophils, red blood cells, megakaryocytes, platelets, dendritic cells. The differentiated cells of the lymphoid lineage are T lymphocytes, B lymphocytes and NK cells.

According to a particular embodiment of the invention, the cells are human CD34⁺ HSPCs from cord blood, or peripheral blood, or bone marrow, or human induced pluripotent stem cells, or embryonic stem cells. The cells of the hematopoietic lineage and the other co-cultured cells (e.g., mesenchymal stem cells, osteoblasts, osteoclasts, osteocytes, fibroblasts, endothelial cells, endothelial progenitor cells, perivascular cells, adipocytes, neural stem cells, neurons, leukocytes, granulocytes, monocytes, lymphocytes, neutrophils, eosinophils, basophils, macrophages, erythrocytes, megakaryocytes, platelets, induced pluripotent stem cells and embryonic stem cells) can carry disease-related genetic mutations or be genetically modified (e.g., plasmid transfection, lentivirus 30 treatment, CRISP/Cas9 system). Genetic modification can be introduced either before or during the 3D culture. The cells of the hematopoietic lineage and the other co-cultured cells can be immortalized (e.g., hematopoietic cell lines, cancer cell lines).

According to a further embodiment of the present invention, the cells of the hematopoietic lineage, including HSPCs and mature blood cells, can be stained with fluorescent antibodies, conjugated or unconjugated, for the visualization of membrane antigens and/or line-specific receptors (e.g., anti-CD3, anti-CD4, anti-CD8, anti-CD34, anti-CD41, anti-CD42a, anti-CD42b, anti-CD45, anti-CD61, anti-CD117, anti-CD235, anti-cMpl) molecular markers (e.g., GFP, TOMATO), fluorescent cytoplasmic tracers (e.g., calcein, carboxyfluorescein succinimidyl ester (CFSE), CellTrac Far Red, CellTracker Deep Red), membrane tracers (e.g. biotinylated or fluorescent lipids and/or phospholipids, 4,4-Difluoro-1,3,5,7,8-Pentamethyl-4-Bora-3°, 4°-Diaza-s-Indacene, Annexin V, fluorescent cholesterol markers), organelles (e.g. fluorescent tracers of mitochondria, endoplasmic reticulum, lysosomes, Golgi apparatus, granules) and/or nucleus (e.g. propidium iodide, 4′,6-diamidino-2-phenylindole, Hoechst), tracers of intracellular calcium movements (e.g. Fura-2 AM, Fluo-3 AM, Fluo-4 AM, Rhod-2 AM, Calcium Green™-AM). Cells can be stained before or after seeding.

For dynamic culture experiments, the inventors tested the usage of a device manufactured using 3D Low Force Stereolithography (LFS) printing technology. The model was created using software for computer-aided design and slicing. The printing was done using long-term non-toxic biocompatible resins. The flow chamber presented multiple units, each housing one single silk scaffold or a multi-layer scaffold, served by inlet and outlet ports for being connected to a perfusion pump system. The final device was optically clear. Optionally, a glass window could be placed at the bottom, or at the top, of the chamber, ideal for live-cell imaging and high-resolution microscopy using long working distance objectives (e.g., confocal microscopy, multiphoton microscopy).

Each unit of the chamber was served by independent peristaltic pumps to drive the perfusion of the cell culture medium from a reservoir to the device. At the outlet, a transfusion bag collected the flow-through. In some embodiments, multiple units can be connected in series and perfused by a single common flow-through. The present invention will now be described for illustrative but non-limiting purposes, according to a preferred embodiment with particular reference to the attached figures, in which:

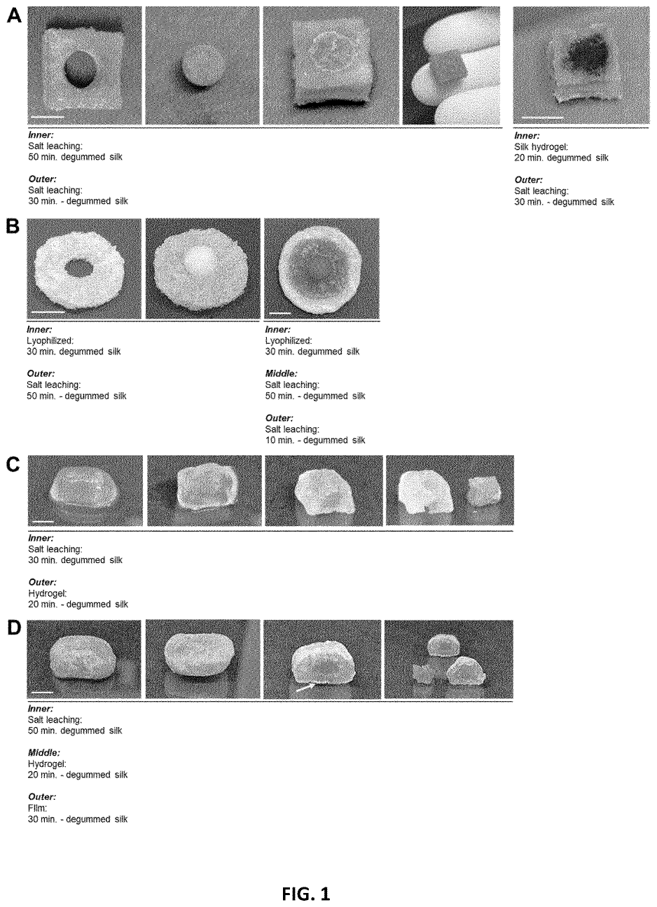

FIG. 1 illustrates exemplary images of the single and multi-layer scaffolds of the invention (scale bars=5 mm). The arrow in Panel D indicates the thin silk film surrounding the hydrogel.

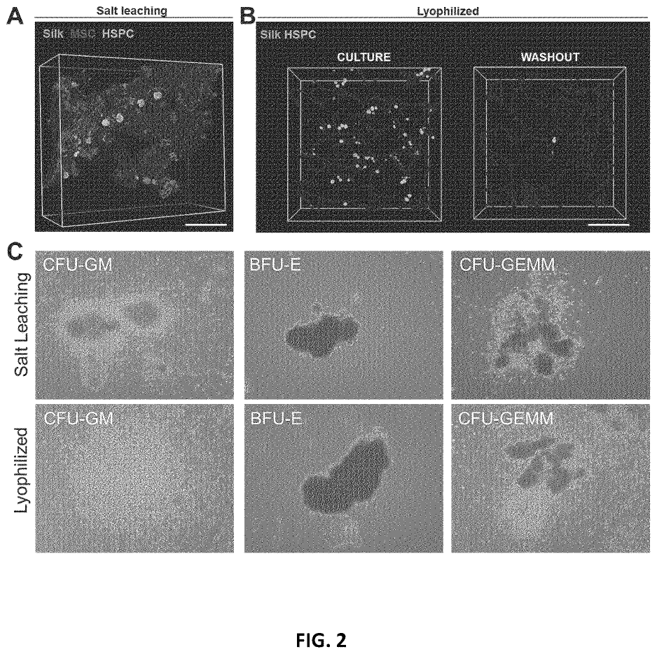

FIG. 2 illustrates 3D HSPC culture into silk scaffolds. Panel A shows the confocal imaging of HSPCs co-cultured with mesenchymal stem cells (MSC) into a solid silk scaffold obtained by salt leaching methods (scale bar=50 μm). Panel B shows the confocal imaging of cells cultured into a solid silk scaffold obtained by lyophilization (scale bar=50 μm). The analysis of the scaffold after the washout of the cells demonstrated effective retrieval of the whole cell population from 3D culture. Panel C shows the representative analysis of colony-forming units from HSPCs retrieved after 3 days of culture into the different silk scaffolds. Comparable results have been obtained from HSPCs retrieved after 1, 3, or 7 days of 3D culture.

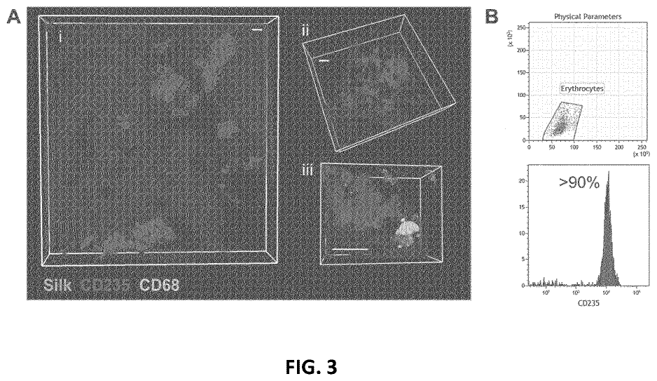

FIG. 3 illustrates HSPCs differentiation into erythrocytes. Panel A shows the confocal microscopy reconstruction of the 3D culture performed into a solid silk scaffold obtained by salt leaching methods (scale bar=50 μm). Panel A iii highlights the migration of CD68⁺ macrophages towards maturing erythrocytes. Panel B shows the flow cytometry analysis of cells retrieved after dynamic perfusion of the system. The analysis demonstrated that CD235^{high} cells are released into the flow through.

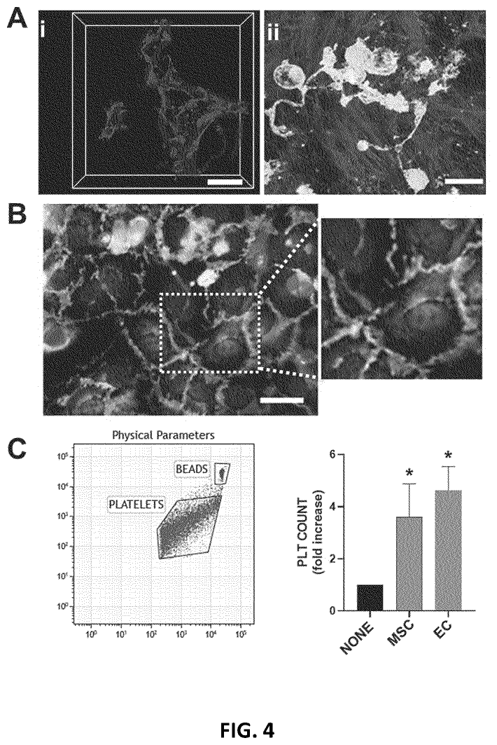

FIG. 4 illustrates HSPCs differentiation into megakaryocytes. Panel A i shows the confocal microscopy reconstruction of the 3D culture performed into the second layer of a solid silk scaffold obtained by salt leaching methods and cultured with mesenchymal stem cells (scale bar=50 μm). Panel A ii shows the confocal microscopy reconstruction of the 3D co-culture mesenchymal stem cells and HSPC in a megakaryocytic differentiation medium. The staining highlights the maturation of CD61⁺ megakaryocytes forming platelets (scale bar=20 μm). Panel B shows the confocal microscopy reconstruction of the culture performed in a solid silk scaffold obtained by salt leaching methods and co-cultured with endothelial cells. The staining highlights the maturation of CD61⁺ megakaryocytes interacting with the endothelium (scale bar=20 μm). Panel C shows the flow cytometry analysis of the numbers of platelets recovered after the dynamic perfusion of the scaffolds. The number of platelets has been estimated by using counting beads.

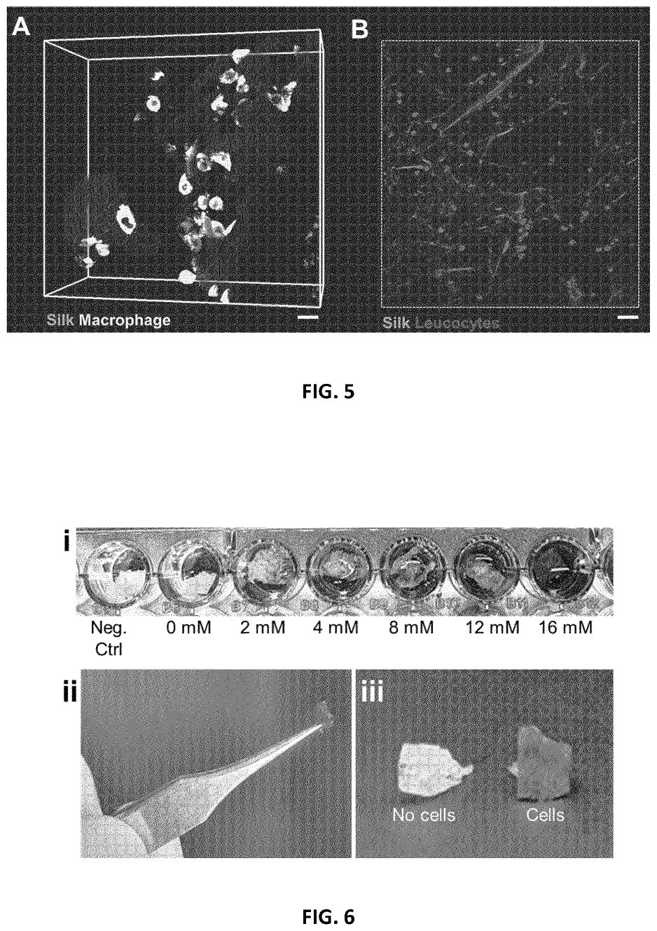

FIG. 5 illustrates mature hematopoietic cells into the silk scaffolds. Panel A shows the confocal microscopy reconstruction of the 3D culture of macrophages performed into a solid silk scaffold obtained by salt leaching methods. Panel B shows the confocal microscopy reconstruction of the 3D culture of leukocytes performed into a solid silk scaffold obtained by lyophilization methods.

FIG. 6, Panel i shows the result of the activation of a colorimetric reaction developed in the HRP functionalized scaffolds incubated with 2-16 mM lactate in the presence of lactate oxidase. Lactate-free incubation was used as a negative control. Panel ii shows the colorimetric reaction activated by the lactate released by the same cells grown inside the 3D scaffold. Panel iii shows the comparison between a scaffold cultured with HSPCs and a scaffold cultured in parallel in the absence of HSPCs, as a negative control.

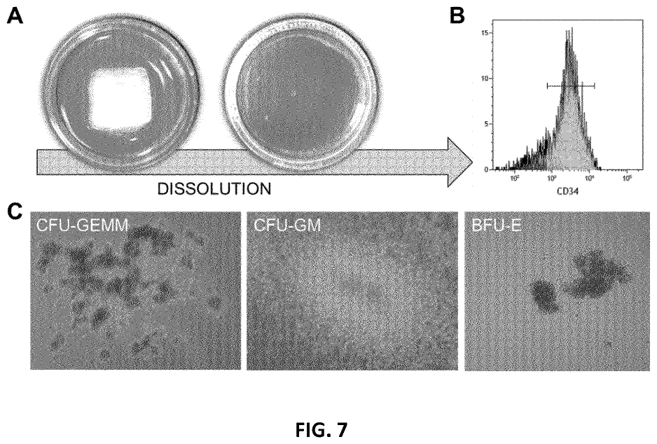

FIG. 7, panel A shows images of the dissolution process of the silk hydrogel obtained with the formulation according to the invention. Dissolution was carried out after 1, 2, 3, 5, and 7 days of culture. The stemness of released HSPCs was confirmed by colony-formation assays. Panel B shows the flow cytometry analysis of CD34⁺ cells retrieved from the silk hydrogel after 3 days of culture into the scaffold. Panel C shows the colony-forming units formed by HSPCs retrieved from the silk hydrogel after 3 days of culture into the scaffold.

The following non-limiting examples are now provided for a better illustration of the invention, in which different silk fibroin-based scaffolds were tested and compared.

### EXAMPLES

### Example 1: Silk Scaffold Preparation

Silk fibroin aqueous solution was obtained from Bombyx mori silkworm cocoons. To prepare pure silk fibroin solution, silkworm cocoons undergo a degumming procedure to separate the silk fibroin fibers from the sericin glue. The molecular weight distribution of silk fibroin chains varies depending on the extraction and purification process utilized to prepare the protein polymer (Sahoo et al., 2020), [10]). Increased degumming time progressively degrades the silk chains resulting in a decrease in the molecular weight from >200 kDa to <90 kDa (experimentally confirmed by Asymmetric Flow Field-Flow Fractionation). Here, dewormed cocoons were boiled for degumming for 10, 20, 30, 40, 50, or 60 minutes in 0.02 M Na₂CO₃ solution at a weight to volume ratio of 5 g to 2 L. The fibers were rinsed for 20 min for three times in ultrapure water and dried overnight. The dried fibers were solubilized for 4 h at 60 °C in LiBr (>9.3 M) at a weight-to-volume ratio of 3 g-12 mL. The solubilized silk solution was dialyzed against distilled water using a Slide-A-Lyzer cassette with a 3500 MW cut-off for three days and changing the water a total of eight times. The silk solution was centrifuged at maximum speed for 10 min to remove large particulates and stored at 4 °C. The concentration of the silk solution was determined by drying a known volume of the solution and massing the remaining solids.

### 1. Solid Silk Scaffolds

NaCl₂ particles (approximately >300 μm in diameter) were sifted into a silk solution (8% w/v) in a ratio of 1 mL silk/2 g salt, within a molding system (e.g., petri dish, silicon chamber). The scaffolds were then placed at room temperature for 48 hours and then soaked in distilled water for 48 hours to leach out the salt particles. The scaffolds were sterilized in 70% ethanol and finally rinsed five times in PBS over 24 hours.

Alternatively, the silk solution (2% w/v) was cast within a molding system and incubated at −80 °C for one day. The frozen solution was then lyophilized for 48-72 hours at −56 °C. The lyophilized scaffolds were subsequently autoclaved for 20 minutes to induce the β-sheet formation and stabilize the silk matrix.

### 2. Hydrogel Silk Scaffold

Silk hydrogels were composed of:

| | | |
|---|---|---|
| silk fibroin | 8% | w/v |
| gelatin or derivatives thereof | 15% | w/v |
| alginic acid or derivatives thereof | 1% | w/v |
| glucose or analogues thereof | 3.5% | w/v |
| Percoll ® | 2.5% | w/v |
| HEPES | 5% | w/v |
| albumin | 0.1% | w/v |

| | |
|---|---|
| saline buffer solution | up to 100% |

wherein the gelatin and alginic acid are present in a reciprocal ratio of about 15:1.

The temperature of the silk hydrogel formulation is 37 °C when the cells are to be added.

Silk hydrogels were produced at 37 °C. After deposition, the silk hydrogel could be crosslinked with a CaCl₂) solution for a minimum of 10 minutes.

The crosslinking solution contains 0.05M CaCl₂) dissolved in a buffer solution containing 150 mM NaCl, 6 mM KCl, 1 mM MgCl₂, 5 mM glucose, 10 mM HEPES.

### 3. Multi-Layer Scaffold Fabrication

Solid and hydrogel silk scaffolds could be assembled into 3D multi-layer tissues that mimic the structure and composition of the different bone marrow microenvironments.

The inner core is a porous solid silk scaffold obtained by salt leaching processing of 50 minutes degummed silk (8%) (FIG. 1A) or by lyophilizing 30 minutes degummed silk (2%) (FIG. 1B).

The second layer of the tissue model can be fabricated with salt leaching methods using 30 minutes degummed silk fibroin (FIG. 1A) or 50 minutes degummed silk fibroin (FIG. 1B). A third layer of the tissue model can be fabricated with salt leaching methods using 10 minutes degummed silk fibroin (FIG. 1B).

The silk hydrogel solution is dispensed at 37 °C around the first solid silk scaffold, and then crosslinked at room temperature (FIG. 1C). Silk fibroin hydrogel was dispensed by manual pipetting.

The hydrogel can also be bio-printed around the solid silk fibroin scaffold through an extrusion-based approach with a nozzle having a caliber of 20G, extrusion pressure of 12 kPa, and printing rate of of 500 mm/min.

A silk film can be layers outside the silk hydrogel (FIG. 1D). The silk film is formed by deeping the multi-layer scaffold into a 2% solution of 30 minutes degummed silk. The system is then soaked into methanol for 2 seconds to allow rapid β-sheet formation and consequent solidification.

### Example 2: HSPC Expansion and Retrieval

To test the ability of the silk scaffolds to support HSPC survival and stemness, human CD34⁺ were cultured inside 30-minute degummed silk scaffolds obtained either by salt leaching (8% silk solution) (FIG. 2A) or lyophilization (2% silk solution) (FIG. 2B), either in static condition or under perfusion with the culture medium for up to 4 weeks with a peristaltic pump:
Salt-leaching: rectangular shape, 200 mm³
Lyophilized: cylindrical shape, 400 mm³

In some experiments, HSCPs have been co-cultured with human mesenchymal stem cells (FIG. 2A). Mesenchymal stem cells were positioned either in the same scaffold of HSPCs or inside the second layer. The 3D culture was monitored and digitally reconstructed by confocal imaging equipped with a temperature-controlled incubator in a humidified atmosphere and 5% CO₂. At the end of the culture, more than 95% of the cells survived in all tested conditions.

HSPC proliferation was analyzed by measuring the number of cells/mm³ by confocal 3D reconstruction. The stemness was determined via colony forming unit (CFU) assays. Cells were retrieved from the silk scaffolds by manual pipetting on days 1, 3, and 7 of 3D culture. The lyophilized silk scaffold allowed a more efficient recovery, with nearly>90% of the cells being pipetted out of the 3D matrix (FIG. 2B). Cells were then plated in a methylcellulose-based medium supplemented with penicillin and streptomycin. Two weeks after plating, colonies were identified according to standard morphological criteria and counted in a blinded fashion (FIG. 2C). A significantly increased number of colonies were generated in methylcellulose assays from the 3D cultures with respect to 2D liquid cultures, performed in parallel. Particularly, the number of BFU-E and CFU-GEMM colonies was significantly higher thus demonstrating retained stemness and differentiation capability.

Differentiation assays were performed in situ. HSPCs were cultured for 7 days in a stem cell medium and then differentiated into erythrocytes or megakaryocytes by switching the culture medium, according to the above-described methods. The imaging of the 3D culture demonstrated efficient cell differentiation into enucleated erythrocytes or platelets, respectively. For static cell cultures, the medium was changed every two days to ensure enough moisture in the system. For dynamic cell cultures, the medium was pumped continuously into the system over the course of the culture, for up to 4 weeks, changing the medium reservoir based on the experimental conditions.

HSPCs co-culture with osteoblasts, seeded into the second layer of the scaffold, demonstrated retained cell proliferation and stem cell profile, even in a medium containing differentiating cytokines (e.g., thrombopoietin). On the opposite, in the presence of mesenchymal stem cells and a megakaryocytic differentiating medium, we observed the proliferation of fully mature megakaryocytes forming increased numbers of platelets with respect to standard liquid cultures.

### Example 3: Erythroid Differentiation and Red Blood Cell Enucleation

HSPCs were seeded into a 30-minute degummed silk scaffold obtained by salt leaching (8% silk solution) and functionalized with fibronectin. The scaffold was filled with enough medium to keep it constantly wet. Cell differentiation was analyzed by means of different microscopy and flow cytometry approaches. We investigated the spatial distribution of cells within the sponge and observed a tendency of developing cellular nests typically resembling the physiological organization of bone marrow erythroblastic island (FIG. 3A i, ii). To investigate the influence of 3D spatial distribution on erythrocyte maturation, we evaluated the expression of lineage-specific surface markers and compared it to 2D cultures. The differentiation patterns were similar in the early-intermediate phases of erythropoiesis, as demonstrated by a comparable percentage of CD235+CD71+ cells. A striking difference could be observed in the terminal phase of maturation. Indeed, the amount of CD235⁺CD71⁻ cells was substantially higher than the 3D cultured cells, suggesting that our silk scaffold effectively supported enucleation. Of note, enucleated CD235⁺CD71⁻ cells were mainly localized at the periphery of the cellular aggregate or next to co-cultured CD68⁺ macrophage (FIG. 3A iii). After two weeks of static culture, the scaffold has been dynamically perfused to assess the effect of laminar flow on erythrocyte terminal differentiation, particularly during the enucleation phase. The flow-through has been collected into a transfusion bag. Flow cytometry analysis demonstrated the recovery of enucleated CD235⁺CD71⁻ cells only while maturing erythroblasts were retained within the scaffold (FIG. 3B). The recovered cell population demonstrated also a significantly decreased expression of alpha4beta1 integrin, which was retained only by immature erythroblasts to favor their adhesion to fibronectin-functionalized silk scaffolds.

### Example 4: Megakaryocyte Differentiation and Platelet Production

Bone marrow cell behavior is regulated by cell-intrinsic and cell-extrinsic forces. Mechanical forces determine mesenchymal stem cells fate (e.g., differentiation into the osteoblast or adipocyte-committed lineage) as well as remodeling of the ECM components. We can regulate mesenchymal stem cell behavior in the niche by changing the mechanical properties of the silk. The scaffolds obtained from the shortest degumming time (10 minutes) exhibited the highest rigidity vs 30- and 50-minute degumming time, as demonstrated by atomic force microscopy-based nanoindentation.

By exploiting these parameters, we fabricated diverse silk tissue models for creating discrete microenvironments able to mimic the different niches that are present in the bone marrow in vivo. The first consisted of a scaffold obtained by the salt leaching approach using 10-minute degummed silk fibroin. The resulting structure confers high mechanical performance and resistance to promote osteogenic induction to human bone marrow mesenchymal stem cells. After two weeks of culture, cells showed an osteoblastic profile as demonstrated by increased nucleation of calcium phosphate, expression of alkaline phosphatase, and deposition of type I collagen. The second scaffold was obtained using 30-minute degummed silk fibroin. This softer model supported mesenchymal stem cell growth and deposition of ECM components.

Co-culturing HSPCs into silk scaffolds containing either mesenchymal stem cells (FIG. 4A), endothelial cells (FIG. 4B), or endothelial-cell derived recombinant proteins (e.g., VEGF, VCAM1, SDF-1alpha), in the presence of a megakaryocytic differentiating medium prolonged megakaryocyte survival and greatly enhanced platelet production in dynamic perfusion. Multi-layer scaffolds were inserted into the 3D printed flow chamber, connected to a syringe pump at the inlet and to a gas-permeable collection bag, containing anticoagulant, at the outlet, and placed into an incubator at 37 °C and 5% CO₂.

The porous silk structure of the scaffolds ensured immediate binding at the interface of the two layers that remained discrete but interconnected entities. Samples have been fed continuously with the culture medium to ensure oxygenation and transport of nutrients through the scaffold. The flow-through is distributed homogeneously within all the layers. Platelet count at the outlet was assessed by flow cytometry by mixing samples with counting beads. Platelets were identified based on the expression of CD41 and CD42b surface markers. The comparison of platelet counts among the different test conditions demonstrated a significant increase in the yield of platelets from megakaryocytes into multi-layers silk scaffold containing mesenchymal stem cells (FIG. 4C) or endothelial cells, with respect to megakaryocytes alone (*p<0.05).

### Example 5: Culture of Mature Blood Cells

Erythrocytes, megakaryocytes, macrophages, or leukocytes were seeded into a 30-minute degummed silk scaffold obtained by salt leaching (8% silk solution) or by lyophilization (2% silk solution) and functionalized with fibronectin. The scaffold was filled with enough medium to keep it constantly wet. Cells were imaged by means of confocal microscopy (FIG. 5).

### Example 6: Bioactive Silk Scaffolds: Dosage of Lactate Released by the Cells During Culture

In order to demonstrate the bioactivity of the silk scaffolds, we functionalized the silk solution with 110 U/mL of horseradish peroxidase (HRP) enzyme before producing the solid silk scaffold or hydrogel, using the same methods described above. The bioactivity was evaluated after several days of culture by activating a reaction leading to chemiluminescence in the presence of luminol and hydrogen peroxide. The chemiluminescent signal was acquired using a chemiluminescence detector. In all the conditions tested, the enzymatic activity was detectable. The functionalized scaffolds were also tested in the presence of hematopoietic progenitors for dosing molecules released during the 3D culture by the cells themselves, without the need for manipulating the scaffold. In particular, the release of lactate, a product of glycolysis, indicative of an active cellular metabolism and whose variation can be indicative of alterations in HSPC proliferation and/or differentiation, was analyzed. For this purpose, a solution of 20 U/mL of lactate oxidase, 600 ng/ml of sodium 3,5-dichloro-2-hydroxybenzenesulfonate (DHBS), and 100 ng/ml of 4-aminoantipyrine (4-AAP) was dispensed onto the scaffolds. Alternatively, the reagents can be added directly to the silk solution before producing the scaffolds. In both cases, the bioactivity of the preparation was confirmed by the activation of a colorimetric reaction following incubation with lactate (2-16 mM) (FIG. 6 i). Lactate-free incubation was used as a negative control. The presence of increasing concentrations of lactate released by the cells was detected during the culture of the bioprinted 3D scaffold (FIG. 6 ii, iii). The specificity of the reaction was confirmed by treating the sample with the lactate dehydrogenase inhibitor.

### Example 7: Dissolution of the Silk Hydrogel

In order to test the possibility of recovering the cells from the silk hydrogel after 3D culture, and using them for functional, biochemical or molecular studies, HSPCs were cultured for 7 days in a stem medium and then incubated in the dissolution solution (according to the invention), at 37 °C for 20 minutes (FIG. 7A).

The cells were then centrifuged at 1,200 rpm for 10 minutes, washed in a physiological solution, and used in the experimental assays.

The morphological parameters and the expression of surface antigens of the stem cell lineage were evaluated by immunofluorescence microscopy and flow cytometry after incubation with specific antibodies (FIG. 7B). Cells were then plated in a methylcellulose-based medium supplemented with penicillin and streptomycin. Two weeks after plating, colonies were identified according to standard morphological criteria and counted in a blinded fashion. The colony-formation assay demonstrated retained stemness and differentiation capability of cells retrieved from the 3D cultures (FIG. 7C).

### REFERENCES

[1] Di Buduo C A, Aguilar A, Soprano P M, Bocconi A, Miguel C P, Mantica G, Balduini A. Latest culture techniques: cracking the secrets of bone marrow to mass-produce erythrocytes and platelets ex vivo. Haematologica. 2021 Apr. 1; 106 (4): 947-957. doi: 10.3324/haematol.2020.262485. PMID: 33472355
[2] Khan A O, Colombo M, Reyat J S, Wang G, Rodriguez-Romera A, Wen W X, Murphy L, Grygielska B, Mahoney C, Stone A, Croft A, Bassett D, Poologasundarampillai G, Roy A, Gooding S, Rayes J, Machlus K R, Psaila B. Human bone marrow organoids for disease modelling, discovery and validation of therapeutic targets in hematological malignancies. bioRxiv 2022.03.14.483815; doi: https://doi.org/10.1101/2022.03.14.483815
[3] Pallotta I, Lovett M, Kaplan D L, Balduini A. Three-dimensional system for the in vitro study of megakaryocytes and functional platelet production using silk-based vascular tubes. Tissue Eng Part C Methods. 2011 December; 17 (12): 1223-32. doi: 10.1089/ten.tec.2011.0134. Epub 2011 Sep. 6. PMID: 21895494
[4] Di Buduo C A, Wray L S, Tozzi L, Malara A, Chen Y, Ghezzi C E, Smoot D, Sfara C, Antonelli A, Spedden E, Bruni G, Staii C, De Marco L, Magnani M, Kaplan D L, Balduini A. Programmable 3D silk bone marrow niche for platelet generation ex vivo and modeling of megakaryopoiesis pathologies. Blood. 2015 Apr. 2; 125 (14): 2254-64. doi: 10.1182/blood-2014-08-595561. Epub 2015 Jan. 9. PMID: 25575540
[5] Di Buduo C A, Wray L, Tozzi L, Chen Y, Kaplan D L, Balduini A. WO2016/022834 A1. Microphysiologic methods and compositions.
[6] Di Buduo C A, Soprano P M, Tozzi L, Marconi S, Auricchio F, Kaplan D L, Balduini A. Modular flow chamber for engineering bone marrow architecture and function. Biomaterials. 2017 November; 146:60-71. doi: 10.1016/j.biomaterials.2017.08.006. Epub 2017 Aug. 8. PMID: 28898758
[7] Tozzi L, Laurent P A, Di Buduo C A, Mu X, Massaro A, Bretherton R, Stoppel W, Kaplan D L, Balduini A. Multi-channel silk sponge mimicking bone marrow vascular niche for platelet production. Biomaterials. 2018 September; 178:122-133. doi: 10.1016/j.biomaterials.2018.06.018. Epub 2018 Jun. 17. PMID: 29920404
[8] Di Buduo C A, Laurent P A, Zaninetti C, Lordier L, Soprano P M, Ntai A, Barozzi S, La Spada A, Biunno I, Raslova H, Bussel J B, Kaplan D L, Balduini C L, Pecci A, Balduini A. Miniaturized 3D bone marrow tissue model to assess response to Thrombopoietin-receptor agonists in patients. Elife. 2021 Jun. 1; 10: e58775. doi: 10.7554/eLife.58775. PMID: 34059198
[9] Di Buduo C A, Kalpan D L, Balduini A. Systems and methods for assessing patient-specific response to thrombopoietin-receptor agonists. WO2021/113830 A1.
[10] Sahoo J K, Choi J, Hasturk O, Laubach I, Descoteaux M L, Mosurkal S, Wang B, Zhang N, Kaplan D L. Silk degumming time controls horseradish peroxidase-catalyzed hydrogel properties. Biomater Sci. 2020 Jul. 28; 8 (15): 4176-4185. doi: 10.1039/d0bm00512f. PMID: 32608410

1. A 3D silk fibroin multi-layer scaffold comprising at least two interconnected silk scaffolds:
i) an inner core made of a porous solid silk fibroin scaffold or of a silk fibroin hydrogel comprising stem cells of the hematopoietic lineage; and
ii) at least one silk fibroin scaffold surrounding the inner core i) made of a silk fibroin hydrogel or porous solid silk fibroin scaffold comprising differentiated or undifferentiated cells, selected from the group consisting of hematopoietic stem cells, hematopoietic progenitor cells, mature blood cells, non-hematopoietic cell types and mixtures thereof.

2. The 3D silk fibroin multi-layer scaffold according to claim 1, wherein the undifferentiated cells of the silk fibroin scaffold ii) are stem and progenitor cells of the hematopoietic lineage, when the 3D silk fibroin multi-layer scaffold further comprises an additional silk layer.

3. The 3D silk fibroin multi-layer scaffold according to claim 1, wherein the stem cells of the hematopoietic lineage are selected from the group consisting of HSPCs, induced pluripotent stem cells, and embryonic stem cells.

4. The 3D silk fibroin multi-layer scaffold according to claim 1, wherein said hematopoietic stem cells, hematopoietic progenitor cells or mature blood cells are selected from the group consisting of leukocytes, granulocytes, monocytes, lymphocytes, neutrophils, eosinophils, basophils, macrophages, erythrocytes, platelets, megakaryocytes, hematopoietic stem cells, induced pluripotent stem cells, and embryonic stem cells, and wherein said non-hematopoietic cell types are selected from the group consisting of osteoblasts, osteoclasts, osteocytes, fibroblasts, endothelial cells, perivascular cells, adipocytes, neurons, mesenchymal stem cells, endothelial progenitor cells, neural stem cell, induced pluripotent stem cells, and embryonic stem cells.

5. The 3D silk fibroin multi-layer scaffold according to claim 1, wherein the porous solid silk fibroin scaffold comprises interconnected pores having a diameter>5 μm.

6. The 3D silk fibroin multi-layer scaffold according to claim 1, wherein said silk fibroin and/or hydrogel scaffold i) or ii), comprises a co-culture with a second differentiated or undifferentiated cell population selected from the group consisting of mesenchymal stem cells, osteoblasts, osteoclasts, osteocytes, fibroblasts, endothelial cells, endothelial progenitor cells, perivascular cells, adipocytes, neural stem cells, neurons, leukocytes, granulocytes, monocytes, lymphocytes, neutrophils, eosinophils, basophils, macrophages, erythrocytes, platelets, megakaryocytes, induced pluripotent stem cells, and embryonic stem cells.

7. The 3D silk fibroin multi-layer scaffold according to claim 1, wherein the silk fibroin and/or hydrogel scaffold i) or ii) is functionalized with one or more bioactive molecules selected from the group consisting of:
components of the extracellular matrix selected from the group consisting of proteoglycans, hyaluronic acid, collagens, elastin, fibronectin, fibrin, fibrinogen, laminins, and thrombospondin;
growth factors, cytokines, and chemokines, interleukins, CSF-1, G-CSF, M-CSF, GM-CSF, SCF, FLT3-L, TPO, EPO, SDF-1α; TGF-β1, TNFα, VEGF, FGF, Notch ligands, WNT, angiopoietin-1, BMP, IGF-2, and fragments or variants thereof;
polyol compounds;
plasma proteins or glycoproteins, selected from the group consisting of albumin, globulins, transferrin, and immunoglobulins;
cellular secretome and cell-derived growth factors;
lipids, cholesterol, and lipoproteins;
biologic fluids, whole blood, bone marrow aspirate, blood serum or plasma, bone marrow serum or plasma, or cell-culture supernatants;
cell-derived interacting motifs, antigens, conjugated or unconjugated antibodies, or their fragments, CDRs or epitopes;
hormones, insulin, glucagon, triiodothyronine, thyroxine, steroid hormones or their antagonists;
drugs or prodrugs, TPO mimetics, TPO receptor agonists, tyrosine kinase receptor agonists, tyrosine kinase receptor inhibitors, Rho kinase inhibitors, kinase inhibitors, receptor antagonists of aryl hydrocarbons, chemotherapeutic agents, monoclonal antibodies, polyclonal antibodies;
nucleic acids, DNA, RNA, siRNA, RNAi and microRNA, plasmids, lentiviruses, CRISPRs;
enzymes, horseradish peroxidase, and/or their substrates or intermediates of enzymatic reactions;
toxins.

8. The 3D silk fibroin multi-layer scaffold according to claim 1, further comprising a thin coating layer of silk fibroin surrounding the outer scaffold to control oxygen distribution.

9. The 3D silk fibroin multi-layer scaffold according to claim 1, wherein the porous solid silk scaffold is obtained by salt leaching processing or by lyophilizing degummed silk fibroin.

10. The 3D silk fibroin multi-layer scaffold according to claim 1, wherein the silk fibroin hydrogel is dispensed around the inner scaffold by pipetting or bio-printing.

11. The 3D silk fibroin multi-layer scaffold according to claim 1, wherein the thin coating layer of silk fibroin surrounding the outer scaffold is formed by dipping, spraying, electrospinning, gel-spinning, or a combination thereof.

12. A 3D porous solid silk fibroin scaffold in a monolayer or multilayer format wherein pores have a diameter>5 μm, obtained by salt leaching processing or by lyophilizing degummed silk fibroin, which is dispersed with stem cells of the hematopoietic lineage alone or in co-culture with differentiated or undifferentiated cells.

13-15. (canceled)

16. A method for expansion and/or differentiation of stem cells of the hematopoietic lineage, comprising culturing the 3D silk fibroin monolayer or multilayer scaffold according to claim 1 in static conditions of perfused into dynamic flow chambers with a cell culture medium comprising at least one nutrient, cytokine, growth factor, hormone, antibody, drug or a combination thereof.

17. A method for the production of a 3D silk fibroin multilayer scaffold comprising:
i) obtaining the porous solid silk scaffold by salt leaching processing or by lyophilizing degummed silk;
ii) dispersing the stem cells of the hematopoietic lineage or progenitor cells into the solid silk scaffolds by manual pipetting; and
iii) mixing the co-cultured differentiated cells within the silk-based hydrogel before dispensing the solution around the solid silk scaffold.

18. The method according to claim 17, further comprising iv) forming a silk coating around the scaffolds by dipping, spraying, electrospinning, gel-spinning, or a combination thereof.

19. The method according to claim 17, further comprising retrieving the cell population from the scaffolds using a solution consisting of sodium citrate, collagenase, proteinase K, liberase and/or alginate lyase, with a source of glucose.

20. The method according to claim 17, wherein said stem cells of the hematopoietic lineage are HSPCs, induced pluripotent stem cells, or embryonic stem cells.

21. The method according to claim 17, wherein the differentiated cells are selected from the group consisting of mammalian, osteoblasts, osteoclasts, osteocytes, fibroblasts, endothelial cells, perivascular cells, adipocytes, neurons, leukocytes, monocytes, lymphocytes, neutrophils, eosinophils, basophils, macrophages, erythrocytes, platelets, and megakaryocytes and the undifferentiated cells are selected from the group consisting of hematopoietic stem cells, mesenchymal stem cells, endothelial progenitor cells, neural stem cell, induced pluripotent stem cells, and embryonic stem cells.
